(19) United States
(12) Patent Application Publication
(10) Pub. No.: US 2021/0257066 A1
(43) Pub. Date: Aug. 19, 2021

(54) MACHINE LEARNING BASED MEDICAL DATA CLASSIFICATION METHOD, COMPUTER DEVICE, AND NON-TRANSITORY COMPUTER-READABLE STORAGE MEDIUM

(71) Applicant: Ping An Technology (Shenzhen) Co., Ltd., Shenzhen (CN)

(72) Inventors: Xianxian CHEN, Shenzhen (CN); Xiaowen RUAN, Shenzhen (CN); Liang XU, Shenzhen (CN)

(73) Assignee: Ping An Technology (Shenzhen) Co., Ltd., Shenzhen (CN)

(21) Appl. No.: 17/165,665

(22) Filed: Feb. 2, 2021

Related U.S. Application Data
(63) Continuation of application No. PCT/CN19/90873, filed on Jun. 12, 2019.

(30) Foreign Application Priority Data
Mar. 7, 2019 (CN) 201910171593.0

(51) Int. Cl. G16H 10/60; G06N 20/00; G16H 50/20; G16H 70/20; G16H 50/70; G06F 40/30; G06F 40/284

(52) U.S. Cl. CPC G16H 10/60; G06N 20/00; G16H 50/20; G06F 40/284; G16H 50/70; G06F 40/30; G16H 70/20

(57) ABSTRACT

A machine learning based medical data classification method is provided. The method includes: a medical data classification request including medical record information is received; a preset medical term base is obtained, and word segmentation is performed on the medical record information according to medical terms in the medical term base to obtain multiple text vectors; features of the multiple text vectors are extracted to obtain multiple text vectors and corresponding feature dimension values; a target classifier is trained with multiple pieces of medical data, and the multiple text vectors and the corresponding feature dimension values are traversed and calculated; until a target node corresponding to the multiple text vectors is traversed, class probabilities corresponding to the multiple text vectors are calculated according to the target node, and a class result corresponding to the medical record information is obtained according to the class probabilities and is pushed to a terminal.

### CROSS-REFERENCE TO RELATED APPLICATIONS

The application is a continuation under 35 U.S.C. § 120 of PCT Application No. PCT/CN2019/090873 filed on Jun. 12, 2019, which claims priority under 35 U.S.C. § 119(a) and/or PCT Article 8 to Chinese Patent Application No. 201910171593.0 filed on Mar. 7, 2019, the disclosures of which are hereby incorporated by reference in their entireties.

### TECHNICAL FIELD

The application relates to the field of computer technology, in particular to a machine learning based medical data classification method and device, a computer device, and a storage medium.

### BACKGROUND

In recent years, the incidence of cancer has been increasing. As a major health problem, early diagnosis and treatment of cancer can significantly increase the survival rate of cancer patients. With the rapid development of computer technology and medical technology, there are some methods for intelligently classifying the massive amounts of medical data. For example, a structured term list of a single medical case is extracted from medical case books, a model of medical case subject is built, and the medical case subject is trained to obtain the corresponding class. Or, an input sample is trained with prior knowledge and then cancer types are classified, which is conducive to reducing the labor intensity of medical personnel.

In the traditional medical data classification method, existing fixed data are mostly used for classification analysis, and the data sources are relatively limited, which makes it impossible to classify and analyze the actual medical record information of users, while the medical record information is mostly complicated and specific medical record analysis and record text. Due to the particularity of medical text, lexical deviations in the medical record information will lead to complete semantic inconsistencies.

### SUMMARY

A machine learning based medical data classification method is performed by a computer device, which includes the following operations.

A medical data classification request sent by a terminal is received, with the medical data classification request including medical record information.

A preset medical term base is obtained, and word segmentation is performed on the medical record information according to medical terms in the medical term base to obtain multiple text vectors.

Features of the multiple text vectors are extracted to obtain multiple text vectors and corresponding feature dimension values.

A target classifier is obtained, and the multiple text vectors and the corresponding feature dimension values are traversed and calculated through multiple neural network nodes of the target classifier. The target classifier is obtained based on the training of multiple pieces of medical data.

Until a target node corresponding to the multiple text vectors is traversed, class probabilities corresponding to the multiple text vectors are calculated according to the target node, and a class result corresponding to the medical record information is obtained according to the class probabilities.

The class result corresponding to the medical record information is pushed to the terminal.

In one of the embodiments, the medical record information includes multiple pieces of text data. The step of word segmentation being performed on the medical record information includes: the preset medical term base is obtained, which includes multiple medical terms; the multiple pieces of text data in the medical record information are matched with the medical term base, a degree of match between the text data in the medical record information and multiple medical terms is calculated, and the text data reaching a preset degree of match is extracted; word segmentation is performed on the medical record information according to the matched text data to obtain multiple pieces of text data after word segmentation; and vector transformation is performed on the multiple pieces of text data after word segmentation to obtain multiple text vectors.

In one of the embodiments, the step that features of the multiple text vectors being extracted to obtain multiple text vectors and corresponding feature dimension values includes: a Term Frequency (TF) and an Inverse Document Frequency (IDF) of the multiple text vectors are calculated; weights of the multiple text vectors are calculated by a preset algorithm based on the TF and the IDF; text vectors whose weights reach a preset threshold are extracted; and feature dimension values corresponding to the text vectors are calculated according to the preset algorithm and the weights.

In one of the embodiments, the step of the target classifier being built includes: multiple pieces of medical data are obtained, and corresponding training set data and verification set data are generated according to the multiple pieces of medical data; clustering analysis is performed on multiple pieces of medical data in the training set data, and a clustering result is obtained; feature extraction is performed on the clustering result to extract multiple feature variables; a preset neural network model is obtained, the training set data is trained through the preset neural network model to obtain the feature dimension values and weights corresponding to the multiple feature variables, and an initial classifier is built according to the feature dimension values and weights corresponding to the multiple feature variables; and the verification set data is used to further train and verify the classifier, and until the amount of the validation set data meeting a preset threshold reaches a preset ratio, training is stopped, and the target classifier required is obtained.

In one of the embodiments, the text includes multiple text sentences, and the multiple text sentences constitute a text block. The step of the multiple text vectors and the corresponding feature dimension values being traversed and calculated through multiple neural network nodes of the target classifier to obtain a class (or classes) corresponding to the multiple text vectors includes: the target classifier is used to calculate the correlation of the multiple text vectors according to the feature dimension values, calculate a text sentence formed in the text according to the correlation, and calculate a sentence vector of the text sentence; a feature of the sentence vector is extracted, and a text block vector is calculated according to the features of multiple sentence vectors; and a probability that the text block vector corresponds to each class is calculated, a class reaching a preset probability value is extracted, and a corresponding class label is added to the text block.

In one of the embodiments, the method further includes: multiple pieces of historical medical data are obtained from a preset database according to a preset frequency; clustering analysis is performed on the multiple pieces of historical medical data, and an analysis result is obtained; features are selected according to the analysis result, and multiple feature variables are obtained; weights of multiple feature variables are calculated according to a preset algorithm.

The target classifier is optimized and adjusted according to the multiple feature variables and the corresponding weights.

A machine learning based medical data classification device includes: a request receiving module, a word segmentation module, a feature extraction module, a data classification module, and a data pushing module.

The request receiving module is configured to receive the medical data classification request sent by the terminal, with the medical data classification request including the medical record information.

The word segmentation module is configured to obtain the preset medical term base, and perform word segmentation to the medical record information according to the medical terms in the medical term base to obtain multiple text vectors.

The feature extraction module is configured to extract the features of the multiple text vectors to obtain multiple text vectors and corresponding feature dimension values.

The data classification module is configured to: obtain the target classifier, and traverse and calculate the multiple text vectors and the corresponding feature dimension values through multiple neural network nodes of the target classifier, with the target classifier being obtained based on the training of multiple pieces of medical data; and until the target node corresponding to the multiple text vectors is traversed, calculate class probabilities corresponding to the multiple text vectors according to the target node, and obtain the class result corresponding to the medical record information according to the class probabilities.

The data pushing module is configured to push the class result corresponding to the medical record information to the terminal.

In one of the embodiments, the word segmentation module is further configured to: obtain the preset medical term base, which includes multiple medical terms; match the multiple pieces of text data in the medical record information with the medical term base, calculate a degree of match between the text data in the medical record information and multiple medical terms, and extract the text data reaching a preset degree of match; perform word segmentation to the medical record information according to the matched text data to obtain multiple pieces of text data after word segmentation; and vectorize multiple pieces of text data after word segmentation to obtain multiple text vectors.

A computer device includes a memory and a processor. The memory stores at least one computer readable instruction. The computer readable instruction is loaded by the processor to perform the following steps.

The medical data classification request sent by the terminal is received, with the medical data classification request including the medical record information.

The preset medical term base is obtained, and word segmentation is performed on the medical record information according to the medical terms in the medical term base to obtain multiple text vectors.

The features of the multiple text vectors are extracted to obtain multiple text vectors and corresponding feature dimension values.

The target classifier is obtained, and the multiple text vectors and the corresponding feature dimension values are traversed and calculated through multiple neural network nodes of the target classifier. The target classifier is obtained based on the training of multiple pieces of medical data.

Until the target node corresponding to the multiple text vectors is traversed, class probabilities corresponding to the multiple text vectors are calculated according to the target node, and the class result corresponding to the medical record information is obtained according to the class probabilities.

The class result corresponding to the medical record information is pushed to the terminal.

A non-transitory computer-readable storage medium stores at least one computer readable instruction. The computer readable instruction is loaded by the processor to perform the following steps.

The medical data classification request sent by the terminal is received, with the medical data classification request including the medical record information.

The preset medical term base is obtained, and word segmentation is performed on the medical record information according to the medical terms in the medical term base to obtain multiple text vectors.

The features of the multiple text vectors are extracted to obtain multiple text vectors and corresponding feature dimension values.

The target classifier is obtained, and the multiple text vectors and the corresponding feature dimension values are traversed and calculated through multiple neural network nodes of the target classifier. The target classifier is obtained based on the training of multiple pieces of medical data.

Until the target node corresponding to the multiple text vectors is traversed, class probabilities corresponding to the multiple text vectors are calculated according to the target node, and the class result corresponding to the medical record information is obtained according to the class probabilities.

The class result corresponding to the medical record information is pushed to the terminal.

### BRIEF DESCRIPTION OF THE DRAWINGS

The drawings needed in the description of the embodiments are simply introduced below. It is apparent for those of ordinary skill in the art that the accompanying drawings in the following description are only some embodiments of the application, and, some other accompanying drawings may also be obtained according to these on the premise of not contributing creative effort.

FIG. 1 is a schematic diagram of an application scenario of a machine learning based medical data classification method in an embodiment.

FIG. 2 is a flowchart of a machine learning based medical data classification method in an embodiment.

FIG. 3 is a flowchart of a step of performing word segmentation to medical record information in an embodiment.

FIG. 4 is a flowchart of a step of building a target classifier in an embodiment.

FIG. 5 is a structural block diagram of a machine learning based medical data classification device in an embodiment.

FIG. 6 is an internal structure diagram of a computer device in an embodiment.

### DETAILED DESCRIPTION OF THE EMBODIMENTS

In order to make the technical solutions and advantages of the application clearer, the application will be further described below in combination with the drawings and the embodiments in detail. It should be understood that the specific embodiments described herein are only adopted to explain the application and not intended to limit the application.

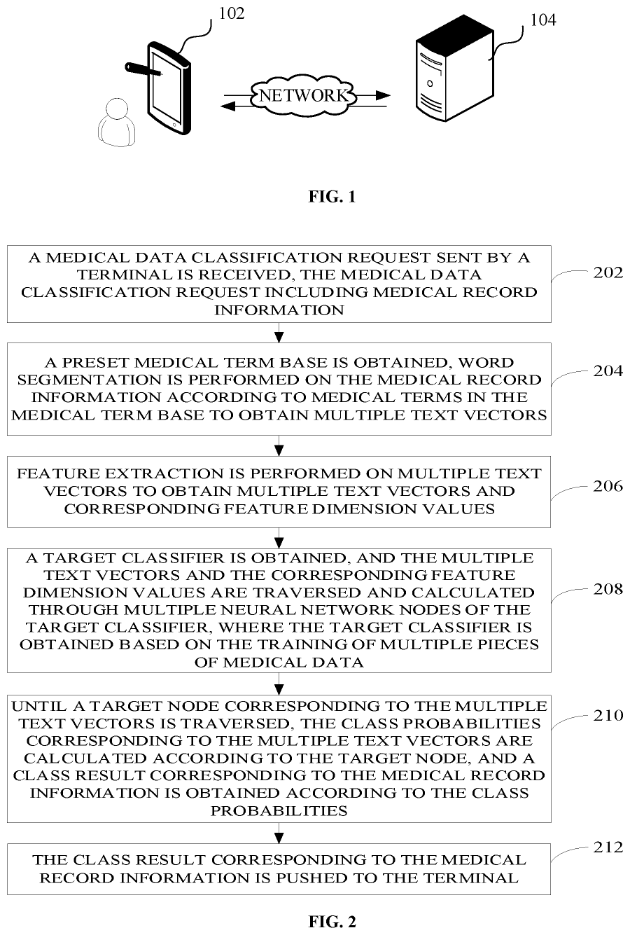

A machine learning based medical data classification method provided by the application may be applied in the application environment as shown in FIG. 1. A terminal 102 communicates with a server 104 through a network. Medical staff may use the corresponding terminal 102 to send a medical data classification request to the server 104, with the medical data classification request including medical record information. After receiving the medical data classification request sent by the terminal 102, the server 104 performs word segmentation on the medical record information to obtain multiple text vectors. Then, the server 104 performs feature extraction on multiple text vectors to obtain multiple text vectors and corresponding feature dimension values. Further, the server 104 obtains a target classifier which is obtained based on the training of multiple pieces of medical data, and performs classified analysis on the obtained multiple text vectors and corresponding feature dimension values through multiple neural network nodes of the target classifier, thereby effectively obtaining a class results corresponding to the medical record information. The server 104 also pushes the class result corresponding to the medical record information to the corresponding terminal 102. The classification accuracy of the medical record information is improved by performing effective word segmentation and feature extraction on the medical record information, and using the classifier trained and built in advance to classify the extracted text data. The terminal 102 may be, but not limited to, a personal computer, a laptop, a smart phone, a tablet computer and a portable wearable device. The server 104 may be realized by an independent server or a cluster of multiple servers.

In one of the embodiments, as shown in FIG. 2, a machine learning based medical data classification method is provided. Illustrated by the application of the method by the server in FIG. 1, the method includes the following steps.

At S202, a medical data classification request sent by a terminal is received, with the medical data classification request including medical record information.

The medical record information may include identity, capital information, medical history information and historical diagnosis information of a patient. When diagnosing a patient, medical staff may use the corresponding terminal to obtain the medical record information of the patient. The medical record information may include information input by the medical staff or the medical record information obtained from a database based on the identity of the patient. After obtaining the medical record information of the patient, the terminal sends a medical data classification request to the server based on the medical record information. The medical data classification request includes the medical record information and the identity.

Further, the server may also obtain historical medical record information from a third-party database based on the patient's identity, for example, the medical record information of the patient in other places, so as to effectively obtain the complete medical record information corresponding to the patient.

At S204, a preset medical term base is obtained, word segmentation is performed on the medical record information according to medical terms in the medical term base to obtain multiple text vectors.

Before performing word segmentation on the medical record information, the server may obtain a large amount of medical data and perform semantic analysis on the large amount of medical data obtained. For example, semantic analysis may be performed on a large amount of medical data through a preset semantic analysis model to obtain multiple types of medical terms. Then, the server uses the medical terms obtained from analysis to generate a medical term base corresponding to multiple types in the medical field.

After receiving the medical data classification request sent by the terminal, the server performs word segmentation on the medical record information. Specifically, the server obtains a preset medical term base. The medical term base includes a large number of medical terms and corresponding vectors. Then, the server matches multiple pieces of text data in the medical record information with multiple medical terms in the medical term base. Specifically, the server may calculate the similarity between the text data in the medical record information and the medical terms through a preset distance algorithm, and then calculate a degree of match between the text data in the medical record information and the medical terms. The server further extracts the text data reaching a preset degree of match. The server performs word segmentation to the medical record information according to the matched text data to obtain multiple pieces of text data after word segmentation. The server further vectorizes multiple pieces of text data after word segmentation, converts the text data into corresponding quantitative information to obtain multiple text vectors corresponding to the multiple pieces of text data.

At S206, feature extraction is performed on multiple text vectors to obtain multiple text vectors and corresponding feature dimension values.

After performing word segmentation on the text vector corresponding to the medical record information to obtain multiple text vectors, the server further performs feature extraction on the text data. The server calculates weights of multiple text vectors after word segmentation according to a preset algorithm. For example, the server may calculate TF values and IDF values of multiple text vectors through a TF-IDF algorithm. TF represents a frequency at which the text vector appears in a document. IDF refers to the measurement of the universal importance of terms. And the server calculates multiple corresponding weights according to the TF values and the IDF values of multiple terms. For example, the weight corresponding to the text vector may be obtained by calculating the product of the TF value and the IDF value, and then the server performs feature extraction on the text vector according to the weight of the text vector, and extracts the text vector reaching a preset threshold.

After extracting the text vector reaching the preset threshold, the server calculates the feature dimension values of multiple text vectors according to the preset algorithm and the weight of the text vector. The feature dimension value may represent a feature dimension to which the text vector belongs. By calculating the weight of the text vector and filtering the text vector according to the weight, feature extraction may be effectively performed on the text vector, and the feature dimension value corresponding to the text vector may be obtained.

At S208, a target classifier is obtained, and the multiple text vectors and the corresponding feature dimension values are traversed and calculated through multiple neural network nodes of the target classifier, where the target classifier is obtained based on the training of multiple pieces of medical data.

At S210, until a target node corresponding to the multiple text vectors is traversed, the class probabilities corresponding to the multiple text vectors are calculated according to the target node, and a class result corresponding to the medical record information is obtained according to the class probabilities.

Before obtaining the target classifier, the server may also build the target classifier in advance or obtain the target classifier by training. Specifically, the server may obtain a large amount of medical data from a local database or a third-party database in advance, and generate corresponding training set data and verification set data based on multiple pieces of medical data. The server vectorizes multiple pieces of field data corresponding to the medical data to obtain the feature vectors corresponding to multiple pieces of text data, and converts the feature vectors into corresponding feature variables. Then, the server uses a preset clustering algorithm to perform clustering analysis on the feature variables corresponding to the training set data, and extracts the feature variable reaching the preset threshold. The server obtains a preset neural network model, trains the training set data through the preset neural network model to obtain the feature dimension values and weights corresponding to the multiple feature variables, and builds an initial classifier according to the feature dimension values and weights corresponding to the multiple feature variables. The server uses the verification set data to further train and verify the classifier, and until the amount of the validation set data meeting the preset threshold reaches a preset ratio, stops training, and obtains the target classifier required.

After performing feature extraction on the text data to obtain multidimensional vectors corresponding to multiple pieces of text data, the server obtains the trained target classifier and inputs multiple text vectors and corresponding dimension feature values into a clear classifier. The target classifier includes several preset neural network layer nodes and corresponding node weights. A loss function is preset through multiple nodes in the target classifier to traverse and calculate multiple text vectors and corresponding dimension feature values until the target node corresponding to multiple text term vectors is obtained, the class probabilities corresponding to multiple text vectors are calculated according to the target node, and the class result corresponding to the text vector is obtained according to the class probabilities, and then a class result corresponding to the medical record information is obtained.

At S212, the class result corresponding to the medical record information is pushed to the terminal.

After classifying the medical record information through the target classifier to obtain the class result corresponding to the medical record information, the server pushes the class result corresponding to the medical record information to the corresponding terminal. By effectively performing word segmentation and feature extraction on the medical record information, and using the target classifier trained and built in advance to classify extracted text information, the classification accuracy of the medical record information can be effectively improved, which can help the medical staff make effective diagnosis according to a pushed class result corresponding to the medical record information, thus effectively improving the diagnostic efficiency of the medical staff.

For example, the medical record information includes the historical medical record information corresponding to the patient, including the description of multiple historical symptoms, historical prescription information, historical diagnosis information, and other data. By using the target classifier trained in advance on the extracted text after multiple screening and text extraction of the medical record information, after classified analysis is performed on all the data in the medical record information of the patient, the class result corresponding to the medical record information is obtained, for example, when the patient is suffering from cancer, the specific type of cancer can be obtained by classifying.

In the machine learning based medical data classification method, after receiving the medical data classification request sent by the terminal, the server performs word segmentation on the medical record information carried in the medical data classification request, so that multiple text vectors can be obtained by performing word segmentation according to the medical field effectively. Then, the server performs feature extraction on multiple text vectors, which can effectively extract multiple text vectors and corresponding feature dimension values. The server further obtains the target classifier which is obtained based on the training of multiple pieces of medical data, traverses and calculates the multiple text vectors and the corresponding feature dimension values through multiple neural network nodes of the target classifier, until the target node corresponding to the multiple text vectors is traversed, calculates the class probabilities corresponding to the multiple text vectors according to the target node, and obtains the class result corresponding to the medical record information according to the class probabilities. In this way, the class result corresponding to the medical record information can be obtained effectively, and the extracted text data can be classified by the classifier trained and built in advance, thus effectively improving the classification accuracy of the medical record information. The server pushes the class result corresponding to the medical record information to the corresponding terminal. In such a manner, it is helpful for the medical staff to make effective decisions according to the pushed class result corresponding to the medical record information, and the processing efficiency of medical data can be improved effectively by classifying the medical record information accurately.

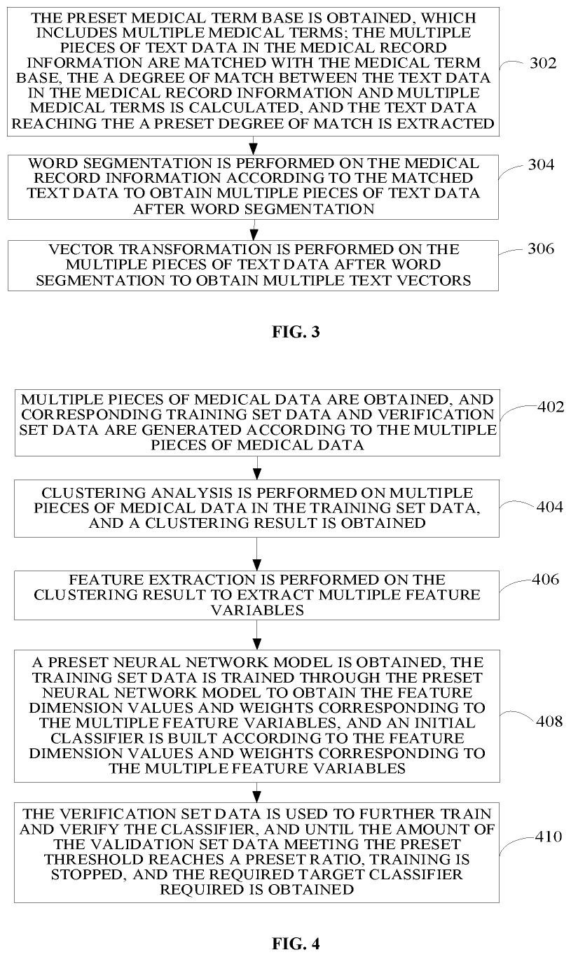

In one of the embodiments, as shown in FIG. 3, the medical record information includes multiple pieces of text data. The step of word segmentation being performed on the medical record information specifically includes the following contents.

At S302, the preset medical term base is obtained, which includes multiple medical terms; the multiple pieces of text data in the medical record information are matched with the medical term base, a degree of match between the text data in the medical record information and multiple medical terms is calculated, and the text data reaching a preset degree of match is extracted.

At S304, word segmentation is performed on the medical record information according to the matched text data to obtain multiple pieces of text data after word segmentation.

At S306, vector transformation is performed on the multiple pieces of text data after word segmentation to obtain multiple text vectors.

Before processing the medical data, the server may build a medical term base in advance. Specifically, the server may obtain a large amount of medical data and perform semantic analysis on the large amount of medical data obtained. For example, semantic analysis may be performed on a large amount of medical data through a preset semantic analysis model to obtain multiple types of medical terms. Then, the server uses the medical terms obtained from analysis to generate a medical term base corresponding to multiple types in the medical field.

The medical staff may use the corresponding terminal to send the medical data classification request to the server, with the medical data classification request including the medical record information. After receiving the medical data classification request sent by the terminal, the server performs word segmentation on the medical record information in the medical data classification request. Specifically, the server obtains the preset medical term base. The medical term base includes a large number of medical terms and corresponding vectors. Then, the server matches multiple pieces of text data in the medical record information with multiple medical terms in the medical term base. Specifically, the server may calculate the similarity between the text data in the medical record information and the medical terms through a preset distance algorithm, and then calculate the degree of match between the text data in the medical record information and the medical terms. The server further extracts the text data reaching the preset degree of match. The server performs word segmentation on the medical record information according to the matched text data to obtain multiple pieces of text data after word segmentation.

The server further vectorizes multiple pieces of text data after word segmentation, converts the text data into corresponding quantitative information to obtain multiple text vectors corresponding to the multiple pieces of text data. For example, Doc2Vec and Word2Vec algorithms may be used to perform word vectorization and paragraph vectorization to multiple pieces of text data after word segmentation, and then the corresponding text vector may be obtained. The text vectors may include a word vector, a word vector, a sentence vector, and so on.

After obtaining the text vectors corresponding to multiple pieces of text data, the server calculates the feature dimension value of the text vector according to the preset algorithm, and performs feature extraction on multiple text vectors to obtain multiple text vectors and corresponding feature dimension values. The server further obtains a preset classifier, and performs classified analysis on the multiple text vectors and corresponding feature dimension values through the classifier, thereby effectively obtaining the class result corresponding to the medical record information. The server also pushes the class result corresponding to the medical record information to the corresponding terminal. By effectively performing word segmentation and feature extraction on the medical record information, and using the classifier trained and built in advance to classify extracted text information, the classification accuracy of the medical record information can be effectively improved, which can help the medical staff make effective diagnosis according to pushed class result corresponding to the medical record information.

In one of the embodiments, the step of feature extraction being performed on multiple pieces of text data to obtain the multidimensional vectors corresponding to multiple text vectors includes: the TF and the IDF of the multiple text vectors are calculated; weights of the multiple text vectors are calculated by the preset algorithm based on the TF and the IDF; text vectors whose weights reach the preset threshold are extracted; and feature dimension values corresponding to the text vectors are calculated according to the preset algorithm and the weights.

The medical staff may use the corresponding terminal to send the medical data classification request to the server, with the medical data classification request including the medical record information. After receiving the medical data classification request sent by the terminal, the server performs word segmentation on the medical record information in the medical data classification request to obtain multiple text vectors.

After obtaining multiple text vectors corresponding to the medical record information, the server calculates the weights of the multiple text vectors after word segmentation according to the preset algorithm. For example, the server may calculate the TF values and the IDF values of multiple text vectors through the TF-IDF algorithm. The TF represents a frequency at which the text vector appears. The IDF may represent the measurement of the universal importance of terms. Multiple corresponding weights are calculated according to the TF values and the IDF values of multiple terms. For example, the weight corresponding to the text vector may be obtained by calculating the product of the TF value and the IDF value.

For example, the TF values of multiple text vectors may be calculated by using the following formula:

$$t {f}_{i , j} = \frac{{n}_{i , j}}{{\sum }_{k} {n}_{k , j}} .$$

The formula for calculating the IDF value of the text vector may be as follows:

$$i d {f}_{i} = l g \frac{\vert D \vert}{\vert \{ j \text{:} {t}_{i} ∈ {d}_{j} \} \vert} .$$

The formula for calculating the weight of the text vector may be as follows:

tfidf_{i,j}=tf_{i,j}×idf_{i,j}.

If there are fewer documents including the text vector t, that is, the smaller n is and the larger IDF is, then it is indicated that the text vector t has a good ability to distinguish classes. If the number of documents including the entry tin a certain type of documents C is m, and the total number of documents including tin other classes is k, it is apparent that the number of documents including t is n=m+k. When m is large, n is also large, and the IDF value obtained according to IDF formula will be small, indicating that this entry does not have strong ability to distinguish class t. If an entry appears frequently in the document of a class, it is indicated that this entry is a good representation of the feature of the text in the class, and the entry has a high weight. By calculating the product of TF and IDF, and calculating the weight of the text vector, the server performs feature extraction on the text vector according to the weight of the text vector, and then extracts the text vector reaching the preset threshold.

After extracting the text vector reaching the preset threshold, the server calculates the feature dimension values of multiple text vectors according to the preset algorithm and the weight of the text vector. The feature dimension value may represent a feature dimension to which the text vector belongs. The text vector may include multiple feature dimensions. After calculating the weight of the text vector, the server may use the weight to calculate the importance of the feature dimension of the text vector, and then obtain the feature dimension value corresponding to the text vector. By calculating the weight of the text vector and filtering the text vector according to the weight, feature extraction may be effectively performed to the text vector, and the feature dimension value corresponding to the text vector may be obtained.

In one of the embodiments, as shown in FIG. 4, before the target classifier is obtained, building the target classifier is also included. The step specifically includes the following contents.

At S402, multiple pieces of medical data are obtained, and corresponding training set data and verification set data are generated according to the multiple pieces of medical data.

Before obtaining the target classifier, the server also needs to build and train the target classifier in advance. Specifically, the server may obtain a large amount of medical data from the local database or the third-party database in advance. The medical data may include medical diagnosis information, clinical data, survey data, etc. The server generates the training set data and the verification set data from a large amount of medical data. The training set data may be the data after manual annotation.

At S404, clustering analysis is performed on multiple pieces of medical data in the training set data, and a clustering result is obtained.

At S406, feature extraction is performed on the clustering result to extract multiple feature variables.

At S408, a preset neural network model is obtained, the training set data is trained through the preset neural network model to obtain the feature dimension values and weights corresponding to the multiple feature variables, and an initial classifier is built according to the feature dimension values and weights corresponding to the multiple feature variables.

At S410, the verification set data is used to further train and verify the classifier, and until the amount of the validation set data meeting the preset threshold reaches a preset ratio, training is stopped, and the target classifier required is obtained.

The server first cleans and preprocesses the medical data in the training set data. Specifically, the server vectorizes multiple pieces of field data corresponding to the medical data to obtain the feature vectors corresponding to multiple pieces of text data, and converts the feature vectors into corresponding feature variables. The server further performs derivative processing on the feature variables to obtain multiple feature variables after processing. For example, a missing value in the feature variable is filled and an abnormal value is extracted and replaced.

Then, the server uses the preset clustering algorithm to perform clustering analysis on the feature variables corresponding to the training set data. For example, the preset clustering algorithm may be a clustering method using k-means (k-means algorithm). The server obtains multiple clustering results by clustering the feature variables for multiple times. The server calculates the similarity among multiple feature variables according to the preset algorithm, and extracts the feature variables whose similarity reaches the preset threshold.

For example, the server may combine the feature variables in multiple clustering results to obtain multiple combined feature variables. A target variable is obtained, and the target variable is used to perform a correlation test on multiple combined feature variables. When the test passes, an interactive label is added to the combined feature variable. The corresponding feature variable is resolved by using the combined feature variable added with the interactive label. The combined feature variable added with the interactive label may be the feature variable reaching the preset threshold. The server extracts the feature variable reaching the preset threshold. By performing feature processing and feature extraction to the feature variables, valuable feature variables may be extracted effectively.

The server obtains a preset machine learning model, for example, the Xgboot machine learning model based on a decision tree. For example, the machine learning model includes multiple neural network models, which may include preset input layer, multiple LSTM layers, dropout layer and output layer. The neural network model includes multiple network nodes, and the abandonment rate of each layer of network nodes may be 0.2.v The LSTM layer of the neural network model includes an activation function and a loss function, and a fully connected artificial neural network output through the LSTM layer may also include a corresponding activation function. The neural network model also includes a calculation method for determining the error, for example, the mean square error algorithm may be used, and an iterative updating method for determining weight parameters, for example, the RMSprop algorithm may be used. The neural network model may also include an ordinary neural network layer for the dimension reduction of output results.

After obtaining the preset neural network model, the server further inputs the medical data in the training set data into the neural network model for learning and training. After the server trains a large amount of medical data in the training set, the corresponding feature dimension values and weights corresponding to multiple feature variables may be obtained, and then the initial classifier is built according to the corresponding feature dimension values and weights corresponding to the multiple feature variables.

After obtaining the initial classifier, the server obtains the verification set data, and trains and verifies the initial classifier through the large amount medical data in the verification set data. Until the amount of the validation set data meeting the preset threshold reaches the preset ratio, training is stopped, and the trained target classifier is obtained. By training and learning a large amount of medical data, the classifier with high forecast accuracy may be built effectively, thus improving the classification accuracy of medical data effectively.

In one of the embodiments, the text includes multiple text sentences, and the multiple text sentences constitute a text block. The step of the multiple text vectors and the corresponding feature dimension values being traversed and calculated through multiple neural network nodes of the target classifier to obtain a class corresponding to the multiple text vectors includes: the target classifier is used to calculate the correlation of the multiple text vectors according to the feature dimension values, calculate a text sentence formed in the text according to the correlation, and calculate a sentence vector of the text sentence; a feature of the sentence vector is extracted, and a text block vector is calculated according to the features of multiple sentence vectors; and a probability that the text block vector corresponds to each class is calculated, a class reaching a preset probability value is extracted, and a corresponding class label is added to the text block.

The medical staff may use the corresponding terminal to send the medical data classification request to the server, with the medical data classification request including the medical record information. After receiving the medical data classification request sent by the terminal, the server performs word segmentation on the medical record information in the medical data classification request to obtain the text vectors corresponding to multiple pieces of text data. The server further performs feature extraction on the text vectors to obtain multiple text vectors and corresponding feature dimension values.

After extracting multiple text vectors and corresponding feature dimension values, the server obtains the target classifier, and takes the multiple text vectors and corresponding feature dimension values as the input of the target classifier. The target classifier includes multiple preset neural network layer nodes and corresponding node weights. Multiple text vectors and corresponding feature dimension values are traversed and calculated through multiple neural network layer nodes in the target classifier. Specifically, the text may include multiple terms and short sentences, that is, text sentences. The text vectors may include term vectors and phrase vectors. The server may first calculate the correlation of multiple text vectors in the text according to the text vector and corresponding dimension feature value, and then calculate the text sentence formed in the text according to the correlation, and calculate a sentence vector corresponding to the text sentence. The server extracts the feature of the sentence vector, and calculates a text block vector according to the features of multiple sentence vectors. The text block includes multiple text sentences. The text block vector may be composed of multiple sentence vectors. The server calculates the probability that the text block vector belongs to each class according to a preset loss function in multiple neural network layer nodes, and inputs multiple text block vectors into the next neural network layer node for calculation until the target node corresponding to multiple text block vectors is obtained, and then calculates the class probabilities corresponding to multiple text block vectors according to the target node, obtains the class result with the highest class probability, thus the class result of multiple text block vectors is obtained. By using the target classifier trained by using a large amount of data to classify the text vectors in the medical record information, the class to which the medical record information belongs can be obtained effectively and accurately, thus the classification accuracy of the medical record information can be improved effectively.

In one of the embodiments, the method further includes: multiple pieces of historical medical data are obtained from a preset database according to a preset frequency; clustering analysis is performed on the multiple pieces of historical medical data, and an analysis result is obtained; features are selected according to the analysis result, and multiple feature variables are obtained; weights of multiple feature variables are calculated according to a preset algorithm; and the target classifier is optimized and adjusted according to the multiple feature variables and the corresponding weights.

After obtaining the target classifier by training, the server may also optimize parameter adjustment of the classifier. Specifically, the server may obtain a large amount of historical medical data from the local database or the third-party database based on a preset frequency. For example, the preset frequency may be one month, three months, six months, etc., while the server may obtain the historical medical data of the past month, three months, and six months. The historical medical data may include medical diagnosis information, clinical data and survey data, etc.

The server first cleans and preprocesses the large amount of historical medical data obtained. Specifically, the server vectorizes multiple pieces of field data corresponding to the historical medical data to obtain the feature variables corresponding to multiple pieces of field data, and performs derivative processing on the feature variables to obtain multiple feature variables after processing. For example, a missing value in the feature variable is filled and an abnormal value is extracted and replaced.

Then, the server uses the preset clustering algorithm to perform clustering analysis on the feature variables corresponding to the training set data. For example, the preset clustering algorithm may be a clustering method using k-means (k-means algorithm). The server obtains multiple clustering results by clustering the feature variables for multiple times. The server calculates the similarity among multiple feature variables according to the preset algorithm, and extracts the feature variables whose similarity reaches the preset threshold.

For example, the server may combine the feature variables in multiple clustering results to obtain multiple combined feature variables. A target variable is obtained, and the target variable is used to perform a correlation test on multiple combined feature variables. When the test passes, an interactive label is added to the combined feature variable. The corresponding feature variable is resolved by using the combined feature variable added with the interactive label. The combined feature variable added with the interactive label may be the feature variable reaching the preset threshold. The server extracts the feature variable reaching the preset threshold. By performing feature processing and feature extraction on the feature variables, valuable feature variables may be extracted effectively.

The server further calculates weights of multiple feature variables according to a preset algorithm, and then optimizes and adjusts the target classifier according to multiple feature variables and corresponding weights. Specifically, the server may adjust the parameters in the target classifier according to multiple feature variables and corresponding weights, thus it can effectively optimize the parameter adjustment of the target classifier.

It should be understood that although the steps in the flowcharts in FIG. 2 to FIG. 4 are shown in order as indicated by the arrows, they are not necessarily performed in the order indicated by the arrows. Unless explicitly stated in the application, there is no strict order in which these steps are performed, and they can be performed in any other order. Furthermore, at least a part of steps in FIG. 2 to FIG. 4 may include multiple sub-steps or multiple stages. These sub-steps or phases are not necessarily performed at the same time, but may be performed at different times. These sub-steps or stages are not necessarily performed in order, but may be performed in turn or alternately with at least a part of other steps or sub-steps or phases of the other steps.

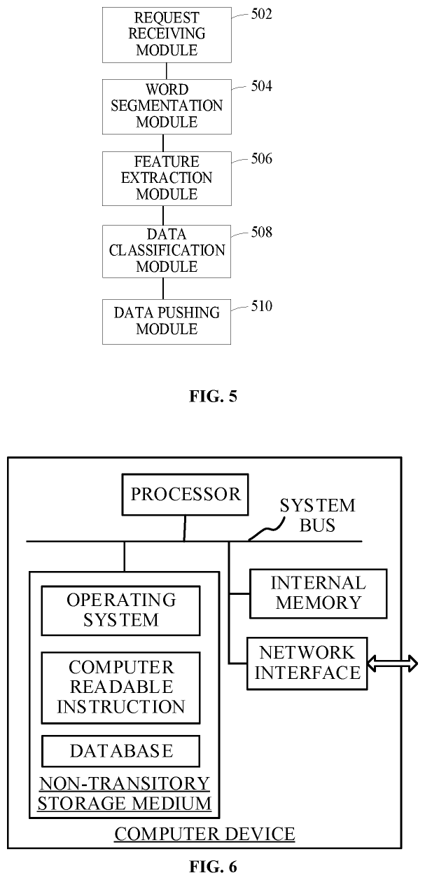

In one of the embodiments, as shown in FIG. 5, a machine learning based medical data classification device is provided, which includes: a request receiving module 502, a word segmentation module 504, a feature extraction module 506, a data classification module 508 and a data pushing module 510.

The request receiving module 502 is configured to receive the medical data classification request sent by the terminal, with the medical data classification request including the medical record information.

The word segmentation module 504 is configured to obtain the preset medical term base, and perform word segmentation on the medical record information according to the medical terms in the medical term base to obtain multiple text vectors.

The feature extraction module 506 is configured to extract the features of the multiple text vectors to obtain multiple text vectors and corresponding feature dimension values.

The data classification module 508 is configured to: obtain the target classifier, and traverse and calculate the multiple text vectors and the corresponding feature dimension values through multiple neural network nodes of the target classifier, with the target classifier being obtained based on the training of multiple pieces of medical data; and until the target node corresponding to the multiple text vectors is traversed, calculate the class probabilities corresponding to the multiple text vectors according to the target node, and obtain the class result corresponding to the medical record information according to the class probabilities.

The data pushing module 510 is configured to push the class result corresponding to the medical record information to the terminal.

In one of the embodiments, the medical record information includes multiple pieces of text data. The word segmentation module 504 is further configured to: obtain the preset medical term base, which includes multiple medical terms; match the multiple pieces of text data in the medical record information with the medical term base, calculate a degree of match between the text data in the medical record information and multiple medical terms, and extract the text data reaching a preset degree of match; perform word segmentation on the medical record information according to the matched text data to obtain multiple pieces of text data after word segmentation; and vectorize multiple pieces of text data after word segmentation to obtain multiple text vectors.

In one of the embodiments, the feature extraction module 506 is further configured to: calculate the TF and the IDF of the multiple text vectors; calculate the weights of the multiple text vectors by the preset algorithm based on the TF and the IDF; extract text vectors whose weights reach a preset threshold; and calculate feature dimension values corresponding to the text vectors according to the preset algorithm and the weights.

In one of the embodiments, the device further includes a target classifier building module, configured to: obtain multiple pieces of medical data, and generate the corresponding training set data and verification set data according to the multiple pieces of medical data; perform clustering analysis on multiple pieces of medical data in the training set data, and obtain a clustering result; perform feature extraction to the clustering result to extract multiple feature variables; obtain a preset neural network model, train the training set data through the preset neural network model to obtain the feature dimension values and weights corresponding to multiple feature variables, and build an initial classifier according to the feature dimension values and weights corresponding to multiple feature variables; and use the verification set data to further train and verify the classifier, and until the amount of the validation set data meeting the preset threshold reaches the preset ratio, stop training, and obtain the target classifier required.

In one of the embodiments, the text includes multiple text sentences, and the multiple text sentences constitute the text block. The data classification module 508 is further configured to use the target classifier to calculate the correlation of the multiple text vectors according to the feature dimension values, calculate the text sentence formed in the text according to the correlation, and calculate the sentence vector of the text sentence; extract a feature of the sentence vector, and calculate the text block vector according to the features of multiple sentence vectors; and calculate a probability that the text block vector corresponds to each class, extract a class reaching the preset probability value, and add the corresponding class label to the text block.

In one of the embodiments, the device further includes a target classifier optimizing module, configured to: obtain multiple pieces of historical medical data from the preset database according to the preset frequency; perform clustering analysis on the multiple pieces of historical medical data, and obtain an analysis result; select the features according to the analysis result, and obtain multiple feature variables; calculate weights of multiple feature variables according to a preset algorithm; and optimize and adjust the target classifier according to the multiple feature variables and the corresponding weights.

For the specific details of the machine learning based medical data classification device, please refer to the details of the machine learning based medical data classification device mentioned above, which will not be repeated here. Each module in the machine learning based medical data classification device may be realized in whole or in part by software, hardware, and their combination. Each above module may be embedded in or independent of a processor in a computer device in the form of hardware, or stored in a memory in the computer device in the form of software, so that the processor may call and perform the operation corresponding to each above module.

In an embodiment, a computer device is provided. The computer device may be a server, and its internal structure may be shown in FIG. 6. The computer device includes a processor, a memory, a network interface, and a database connected through a system bus. The processor of the computer device is used to provide computing and control capabilities. The memory of the computer device includes a non-transitory storage medium and an internal memory. The non-transitory storage medium stores an operating system, a computer readable instruction and a database. The internal memory provides an environment for the operation of the operating system and the computer readable instruction. The database of the computer device is used to store the medical data, the medical record information, and other data. The network interface of the computer device is used to communicate with an external terminal through a network connection. The computer readable instruction, when executed by the processor, implements the steps of the machine learning based medical data classification method in any embodiment of the application.

Those of ordinary skill in the art may understand that the structure shown in FIG. 6 is only a block diagram of part of the structure related to the solutions of the application and does not constitute a limitation on the computer device applied to the solutions of the application. Specifically, the computer device may include more or fewer parts than shown in the figures, or some combination of parts, or a different arrangement of parts.

Those ordinary skilled in the art may understand that all or a part of flows of the method in the above embodiments may be completed by related hardware instructed by a computer readable instruction. The computer readable instruction may be stored in a non-transitory computer readable storage medium. When executed, the computer readable instruction may include the flows in the embodiments of the method. Any reference to memory, storage, database or other media used in each embodiment provided in the application may include non-transitory and/or transitory memories. The non-transitory memories may include a Read-Only Memory (ROM), a Programmable Read-Only Memory (PROM), an Electrically Programmable Read-Only Memory (EPROM), an Electrically Erasable Programmable Read-Only Memory (EEPROM) or a flash memory. The transitory memories may include a Random Access Memory (RAM) or an external cache memory. As an illustration rather than a limitation, the RAM is available in many forms, such as Static RAM (SRAM), Dynamic RAM (DRAM), Synchronous DRAM (SDRAM), Double Data Rate SDRAM (DDRAM), Enhanced SDRAM (ESDRAM), Synch-link DRAM (SLDRAM), Rambus Direct RAM (RDRAM), Direct Memory Bus Dynamic RAM (DRDRAM), and Memory Bus Dynamic RAM (RDRAM).

The technical features of the above embodiments may be combined at random. To make the description concise, not all possible combinations of these technical features of the above embodiments are described, however, all the combinations of these technical features shall fall within the scope of the specification, as long as there is no contradiction in the combinations of these technical features.

The above embodiments only express several implementation modes of the application. The description of these embodiments is more specific and detailed, but cannot be understood as a limitation to the claimed scope of the disclosure. It should be pointed out that those of ordinary skill in the art can also make several improvements and modifications without departing from the concept of the application, and these improvements and modifications should fall within the scope of protection of the application. Therefore, the protection scope of the application is subject to the attached claims.

What is claimed is:

1. A machine learning based medical data classification method, comprising:
receiving a medical data classification request sent by a terminal, wherein the medical data classification request comprises medical record information;
obtaining a preset medical term base, and performing word segmentation on the medical record information according to medical terms in the medical term base to obtain a plurality of text vectors;
extracting features of the plurality of text vectors to obtain a plurality of text vectors and corresponding feature dimension values;
obtaining a target classifier, and traversing and calculating the plurality of text vectors and the corresponding feature dimension values through a plurality of neural network nodes of the target classifier, wherein the target classifier is obtained based on training of a plurality of pieces of medical data;
until a target node corresponding to the plurality of text vectors is traversed, calculating class probabilities corresponding to the plurality of text vectors according to the target node, and obtaining a class result corresponding to the medical record information according to the class probabilities; and
pushing the class result corresponding to the medical record information to the terminal.

2. The method as claimed in claim 1, wherein the medical record information comprises a plurality of pieces of text data, and wherein performing word segmentation on the medical record information comprises:
obtaining the preset medical term base, which comprises a plurality of medical terms;
matching the plurality of pieces of text data in the medical record information with the medical term base, calculating a degree of match between the text data in the medical record information and a plurality of medical terms, and extracting the text data that reach a preset degree of match;
performing word segmentation on the medical record information according to the matched text data to obtain a plurality of pieces of text data after word segmentation; and
vectorizing the plurality of pieces of text data after word segmentation to obtain a plurality of text vectors.

3. The method as claimed in claim 1, wherein extracting the features of the plurality of text vectors to obtain the plurality of text vectors and the corresponding feature dimension values comprises:
calculating a Term Frequency (TF) and an Inverse Document Frequency (IDF) of the plurality of text vectors;
calculating weights of the plurality of text vectors by a preset algorithm based on the TF and the IDF;
extracting text vectors whose weights reach a preset threshold; and
calculating feature dimension values corresponding to the text vectors according to the preset algorithm and the weights.

4. The method as claimed in claim 1, wherein obtaining the target classifier comprises:
obtaining a plurality of pieces of medical data, and generating corresponding training set data and verification set data according to the plurality of pieces of medical data;
performing clustering analysis on a plurality of pieces of medical data in the training set data, and obtaining a clustering result;
performing feature extraction on the clustering result to extract a plurality of feature variables;
obtaining a preset neural network model, training the training set data through the preset neural network model to obtain feature dimension values and weights corresponding to the plurality of feature variables, and building an initial classifier according to the feature dimension values and weights corresponding to the plurality of feature variables; and
using the verification set data to further train and verify the classifier, and until an amount of validation set data that meets a preset threshold reaches a preset ratio, stopping training, and obtaining the target classifier.

5. The method as claimed in claim 1, wherein the text comprises a plurality of text sentences, the plurality of text sentences constitute a text block, and traversing and calculating the plurality of text vectors and the corresponding feature dimension values through the plurality of neural network nodes of the target classifier to obtain a class corresponding to the plurality of text vectors comprises:
using the target classifier to calculate correlation of the plurality of text vectors according to the feature dimension values, calculate a text sentence formed in the text according to the correlation, and calculate a sentence vector of the text sentence;
extracting a feature of the sentence vector, and calculating a text block vector according to the features of plurality of sentence vectors; and
calculating a probability that the text block vector corresponds to each class, extracting a class reaching a preset probability value, and adding a corresponding class label to the text block.

6. The method as claimed in claim 2, wherein the text comprises a plurality of text sentences, the plurality of text sentences constitute a text block, and traversing and calculating the plurality of text vectors and the corresponding feature dimension values through the plurality of neural network nodes of the target classifier to obtain a class corresponding to the plurality of text vectors comprises:
using the target classifier to calculate correlation of the plurality of text vectors according to the feature dimension values, calculate a text sentence formed in the text according to the correlation, and calculate a sentence vector of the text sentence;
extracting a feature of the sentence vector, and calculating a text block vector according to the features of plurality of sentence vectors; and
calculating a probability that the text block vector corresponds to each class, extracting a class reaching a preset probability value, and adding a corresponding class label to the text block.

7. The method as claimed in claim 3, wherein the text comprises a plurality of text sentences, the plurality of text sentences constitute a text block, and traversing and calculating the plurality of text vectors and the corresponding feature dimension values through the plurality of neural network nodes of the target classifier to obtain a class corresponding to the plurality of text vectors comprises:
using the target classifier to calculate correlation of the plurality of text vectors according to the feature dimension values, calculate a text sentence formed in the text according to the correlation, and calculate a sentence vector of the text sentence;
extracting a feature of the sentence vector, and calculating a text block vector according to the features of plurality of sentence vectors; and
calculating a probability that the text block vector corresponds to each class, extracting a class reaching a preset probability value, and adding a corresponding class label to the text block.

8. The method as claimed in claim 1, further comprising:
obtaining a plurality of pieces of historical medical data from a preset database according to a preset frequency;
performing clustering analysis on the plurality of pieces of historical medical data, and obtaining an analysis result;
selecting features according to the analysis result, and obtaining a plurality of feature variables;
calculating weights of a plurality of feature variables according to a preset algorithm; and
optimizing and adjusting the target classifier according to the plurality of feature variables and the corresponding weights.

9. A computer device, comprising:
a memory and a processor, wherein the memory stories at least one computer readable instruction, wherein the at least one computer readable instruction is executable by the processor to perform:
receiving a medical data classification request sent by a terminal, wherein the medical data classification request comprises medical record information;
obtaining a preset medical term base, and performing word segmentation on the medical record information according to medical terms in the medical term base to obtain a plurality of text vectors;
extracting features of the plurality of text vectors to obtain a plurality of text vectors and corresponding feature dimension values;
obtaining a target classifier, and traversing and calculating the plurality of text vectors and the corresponding feature dimension values through a plurality of neural network nodes of the target classifier, wherein the target classifier is obtained based on training of a plurality of pieces of medical data;
until a target node corresponding to the plurality of text vectors is traversed, calculating class probabilities corresponding to the plurality of text vectors according to the target node, and obtaining a class result corresponding to the medical record information according to the class probabilities; and
pushing the class result corresponding to the medical record information to the terminal.

10. The computer device as claimed in claim 9, wherein the medical record information comprises a plurality of pieces of text data, and wherein to perform performing word segmentation on the medical record information, the at least one computer readable instruction, when executed by the processor, causes the processor to perform:
obtaining the preset medical term base, which comprises a plurality of medical terms;
matching the plurality of pieces of text data in the medical record information with the medical term base, calculating a degree of match between the text data in the medical record information and a plurality of medical terms, and extracting the text data reaching a preset degree of match;
performing word segmentation on the medical record information according to the matched text data to obtain a plurality of pieces of text data after word segmentation; and
performing vector transformation on the plurality of pieces of text data after word segmentation to obtain a plurality of text vectors.

11. The computer device as claimed in claim 9, wherein to perform extracting the features of the plurality of text vectors to obtain the plurality of text vectors and the corresponding feature dimension values, the at least one computer readable instruction, when executed by the processor, causes the processor to perform:
calculating a Term Frequency (TF) and an Inverse Document Frequency (IDF) of the plurality of text vectors;
calculating weights of the plurality of text vectors by a preset algorithm based on the TF and the IDF;
extracting text vectors whose weights reach a preset threshold; and
calculating the feature dimension values corresponding to the text vectors according to the preset algorithm and the weights.

12. The computer device as claimed in claim 9, wherein to perform obtaining the target classifier, the at least one computer readable instruction, when executed by the processor, causes the processor to perform:
obtaining a plurality of pieces of medical data, and generating corresponding training set data and verification set data according to the plurality of pieces of medical data;
performing clustering analysis on a plurality of pieces of medical data in the training set data, and obtaining a clustering result;
performing feature extraction on the clustering result to extract a plurality of feature variables;
obtaining a preset neural network model, training the training set data through the preset neural network model to obtain feature dimension values and weights corresponding to the plurality of feature variables, and building an initial classifier according to the feature dimension values and weights corresponding to the plurality of feature variables; and
using the verification set data to further train and verify the classifier, and until an amount of validation set data that meets a preset threshold reaches a preset ratio, stopping training, and obtaining the target classifier.

13. The computer device as claimed in claim 9, wherein the text comprises a plurality of text sentences, and the plurality of text sentences constitute a text block, and wherein to perform traversing and calculating the plurality of text vectors and the corresponding feature dimension values through the plurality of neural network nodes of the target classifier, the at least one computer readable instruction, when executed by the processor, causes the processor to perform:
using the target classifier to calculate the correlation of the plurality of text vectors according to the feature dimension values, calculate a text sentence formed in the text according to the correlation, and calculate a sentence vector of the text sentence;
extracting a feature of the sentence vector, and calculating a text block vector according to the features of a plurality of sentence vectors; and
calculating a probability that the text block vector corresponds to each class, extracting a class reaching a preset probability value, and adding a corresponding class label to the text block.

14. The computer device as claimed in claim 9, wherein the at least one computer readable instruction, when executed by the processor, further causes the processor to perform:
obtaining a plurality of pieces of historical medical data from a preset database according to a preset frequency;
performing clustering analysis on the plurality of pieces of historical medical data, and obtaining an analysis result;
selecting features according to the analysis result, and obtaining a plurality of feature variables;
calculating weights of a plurality of feature variables according to a preset algorithm; and
optimizing and adjusting the target classifier according to the plurality of feature variables and the corresponding weights.

15. A non-transitory computer-readable storage medium that stores at least one computer readable instruction, wherein the at least one computer readable instruction, when executed by a processor, causes the processor to perform:
receiving a medical data classification request sent by a terminal, wherein the medical data classification request comprises medical record information;
obtaining a preset medical term base, and performing word segmentation on the medical record information according to medical terms in the medical term base to obtain a plurality of text vectors;
extracting features of the plurality of text vectors to obtain a plurality of text vectors and corresponding feature dimension values;
obtaining a target classifier, and traversing and calculating the plurality of text vectors and the corresponding feature dimension values through a plurality of neural network nodes of the target classifier, wherein the target classifier is obtained based on training of a plurality of pieces of medical data;
until a target node corresponding to the plurality of text vectors is traversed, calculating class probabilities corresponding to the plurality of text vectors according to the target node, and obtaining a class result corresponding to the medical record information according to the class probabilities; and
pushing the class result corresponding to the medical record information to the terminal.

16. The storage medium as claimed in claim 15, wherein the medical record information comprises a plurality of pieces of text data, and wherein to perform performing word segmentation on the medical record information, the at least one computer readable instruction, when executed by the processor, causes the processor to perform:
obtaining the preset medical term base, which comprises a plurality of medical terms;
matching the plurality of pieces of text data in the medical record information with the medical term base, calculating a degree of match between the text data in the medical record information and a plurality of medical terms, and extracting the text data reaching a preset degree of match;
performing word segmentation on the medical record information according to the matched text data to obtain a plurality of pieces of text data after word segmentation; and
performing vector transformation on the plurality of pieces of text data after word segmentation to obtain a plurality of text vectors.

17. The storage medium as claimed in claim 15, wherein to perform extracting the features of the plurality of text vectors to obtain the plurality of text vectors and the corresponding feature dimension values, the at least one computer readable instruction, when executed by the processor, causes the processor to perform:
calculating a Term Frequency (TF) and an Inverse Document Frequency (IDF) of the plurality of text vectors;
calculating weights of the plurality of text vectors by a preset algorithm based on the TF and the IDF;
extracting text vectors whose weights reach a preset threshold; and
calculating feature dimension values corresponding to the text vectors according to the preset algorithm and the weights.

18. The storage medium as claimed in claim 15, wherein to perform obtaining the target classifier, the at least one computer readable instruction, when executed by the processor, causes the processor to perform:
obtaining a plurality of pieces of medical data, and generating corresponding training set data and verification set data according to the plurality of pieces of medical data;
performing clustering analysis on a plurality of pieces of medical data in the training set data, and obtaining a clustering result;
performing feature extraction on the clustering result to extract a plurality of feature variables;
obtaining a preset neural network model, training the training set data through the preset neural network model to obtain feature dimension values and weights corresponding to the plurality of feature variables, and building an initial classifier according to the feature dimension values and weights corresponding to the plurality of feature variables; and
using the verification set data to further train and verify the classifier, and until an amount of validation set data that meets a preset threshold reaches a preset ratio, stopping training, and obtaining the target classifier.

19. The storage medium as claimed in claim 15, wherein the text comprises a plurality of text sentences, and the plurality of text sentences constitute a text block, and wherein to perform traversing and calculating the plurality of text vectors and the corresponding feature dimension values through the plurality of neural network nodes of the target classifier, the at least one computer readable instruction, when executed by the processor, causes the processor to perform:
using the target classifier to calculate correlation of the plurality of text vectors according to the feature dimension values, calculate a text sentence formed in the text according to the correlation, and calculate a sentence vector of the text sentence;
extracting a feature of the sentence vector, and calculating a text block vector according to the features of a plurality of sentence vectors; and
calculating a probability that the text block vector corresponds to each class, extracting a class reaching a preset probability value, and adding a corresponding class label to the text block.

20. The storage medium as claimed in claim 15, wherein the at least one computer readable instruction, when executed by the processor, further causes the processor to perform:
obtaining a plurality of pieces of historical medical data from a preset database according to a preset frequency;
performing clustering analysis on the plurality of pieces of historical medical data, and obtaining an analysis result;
selecting features according to the analysis result, and obtaining a plurality of feature variables;
calculating weights of a plurality of feature variables according to a preset algorithm; and
optimizing and adjusting the target classifier according to the plurality of feature variables and the corresponding weights.
